(19) United States
(12) Patent Application Publication
(10) Pub. No.: US 2023/0173180 A1
(43) Pub. Date: Jun. 8, 2023

(54) INJECTION DEVICE

(71) Applicant: Owen Mumford Limited, Woodstock, Oxfordshire (GB)

(72) Inventor: Toby COWE, Woodstock, Oxfordshire (GB)

(21) Appl. No.: 17/923,828

(22) PCT Filed: May 10, 2021
(86) PCT No.: PCT/IB2021/053967

§ 371 Date: Nov. 7, 2022

(30) Foreign Application Priority Data
May 7, 2020 (GB) 2006835.9

(51) Int. Cl. A61M 5/20; A61M 5/50

(52) U.S. Cl. CPC A61M 5/20; A61M 5/5086; A61M 2005/206; A61M 2205/6072

(57) ABSTRACT

Aspects of the present invention relate to an injection device configured to receive a medicament container. The injection device comprises movable means, such that before injection an information code is not visible and such that during or after injection the movable means moves to allow the code to be visible.

### TECHNICAL FIELD

The present disclosure relates to an injection device. In particular, it relates to an injection device configured to selectively display an information code.

### BACKGROUND

It is known to provide information codes, such as a bar code, on a medicament container in order that information relating to any medicament in the container can be ascertained. This information includes, medicament type, batch codes for example.

### SUMMARY OF THE INVENTION

Aspects and embodiments of the invention provide an injection device as claimed in the appended claims.

According to an aspect of the present invention there is provided an injection device for receiving a medicament container comprising movable means, the injection device being configured such that before injection an information code is not visible and such that during or after injection the movable means moves to allow the code to be visible. This means that information relating to the medicament can only be uploaded once injection has been completed allowing confirmation of the medicament delivery to happen.

The injection device may have a housing including a transparent area, wherein the movable means moves from a position where the information code cannot be viewed through the transparent area to a position where the information code can be viewed through the transparent area.

For example, the movable means may be a plunger. The information code is situated on the plunger such that the plunger moves from a start position in which the information code cannot be viewed to a final position where the information code can be viewed through the transparent area.

The transparent area may be, for example, a transparent area in a label applied to a housing or a transparent area in the material of the housing.

The movable means may be a needle shroud and the information code may be provided on the medicament container. In this instance the needle shroud moves from a position where it covers the information code in order that the information code cannot be viewed to a position where the information code can be viewed either through a transparent portion of the housing or outside the housing if not all of the medicament container is found within the housing.

In an alternative embodiment the movable means comprises a needle shroud, the information code being carried on the needle shroud, wherein the needle shroud moves from a position where it is at least partially contained within a housing of the injection device and the information code is not visible to a position where the needle shroud extends from the housing and the information code is visible.

The injection device comprises an auto-injector or a manual syringe.

The information code may comprise a bar code or a QR code.

### BRIEF DESCRIPTION OF THE DRAWINGS

One or more embodiments of the invention will now be described, by way of example only, with reference to the accompanying drawings, in which:

FIGS. 1a to 1e shows an embodiment of an injection device in accordance with the present invention;

FIGS. 2 and 3 show an alternative example of the injection device of FIGS. 1a to 1e;

FIGS. 4a to 4e show an alternative embodiment of an injection device in accordance with the present invention.

### DETAILED DESCRIPTION

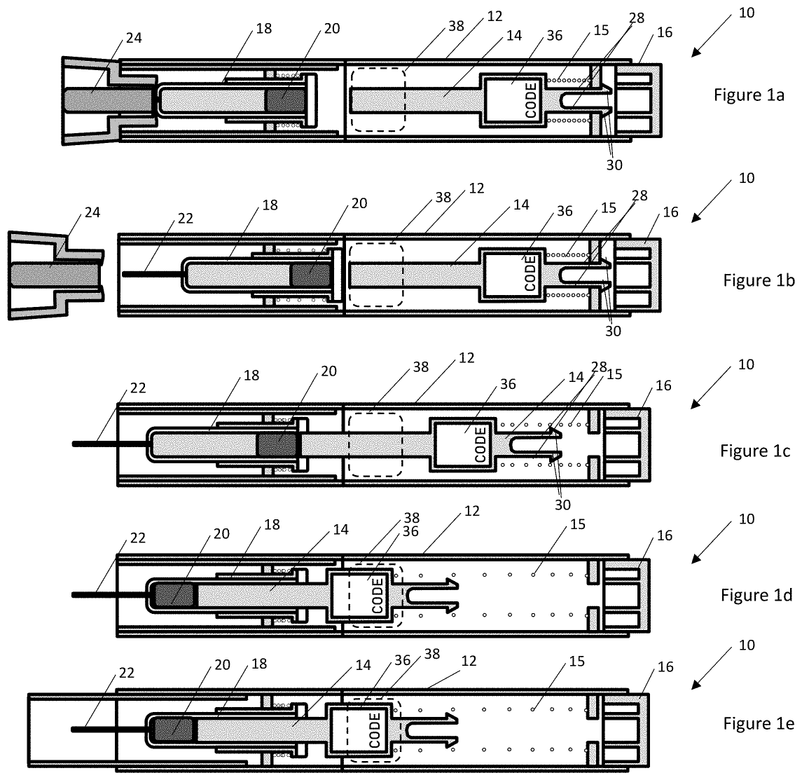

An injection device according to an embodiment of the present invention is described herein with reference to FIGS. 1a to 1e.

With reference to FIG. 1, there is provided an auto-injector 10. The auto-injector 10 comprises a housing 12, a plunger 14, a spring 15 and a button 16. The housing is configured to receive a container 18 which has an elastomeric piston 20 at one end and a needle 22 at the other end. Contained within the container 18 is a medicament. In the auto-injector's initial configuration it is provided with a needle shield 24 which may be soft or rigid. The needle shield 24 may be provided with an additional, separate remover 26 to assist its removal although this is not essential.

The plunger 14 is provided with two flexible arms 28 having projections 30 which interact and are retained by projections 32 from the housing 12. A spring 34 is retained in compression between a portion of the housing and a part of the auto-injector. Additionally the plunger 14 is provided with a region on which an information code 36 is provided. The housing 12 is provided with a window 38 through which the internal part of the auto-injector can be seen as discussed below.

Prior to use of the auto-injector 10 the needle shield 24 is removed from the needle 22 of the device as illustrated in FIG. 1b. The auto-injector is introduced to the skin of a body to be injected (not shown). Once the auto-injector 10 has been introduced to the skin of the body to be injected the button 16 is pressed by a user.

As shown in FIG. 1c pressing the button causes the two flexible arms 28 to move inwards thereby causing the projections 30 of the arms 28 to no longer be retained by the projections 32 of the housing 12. This allows the spring 34 which has been retained in a compression state to exert a force on the plunger 14. The movement of the plunger 14 causes consequent movement of the piston 20 of the medicament container 18 and thereby causes medicament to be expelled from the container 18 through the needle 22 as it moves to the position illustrated in FIG. 1d.

Movement of the plunger 14 through the auto-injector 10 means that the information code 36 present on the plunger 14 also moves towards the distal end of the auto-injector 10. The window 38 of the housing 12 is positioned such that the information code 36 is visible through the window 38 when the auto-injector has completed the injection of medicament from the container. This allows information relating to the medicament to only be read after the medicament has been delivered. Once the information code can be seen through the window 38 it can be scanned by a user. This may be achieved using any suitable device including but not limited to, a smartphone, a camera, a webcam or specialist scanning equipment. The information may be received directly by an app, for example on a smartphone or through a website. Uploading this information allows recordal of completion of delivery of the drug. This may be useful for example, to control access to drugs.

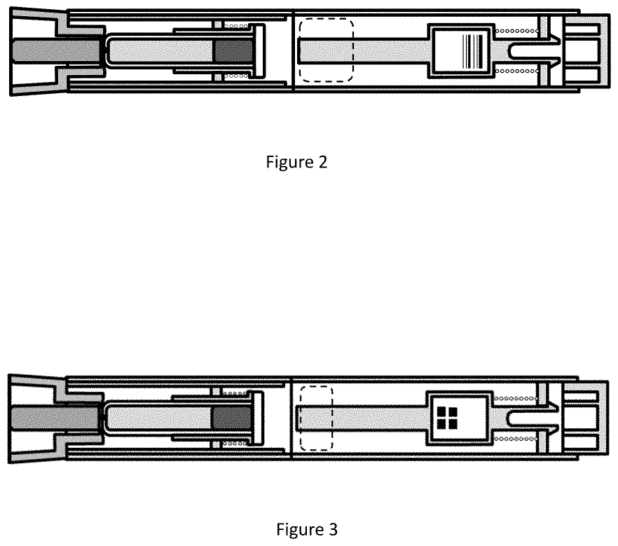

It will be understood that the information code may be any suitable code configured to include information. For example, the information code may be a bar code as illustrated in FIG. 2 or a QR code as illustrated in FIG. 3. Information contained in the information code, bar code or QR code may be, for example, batch number, a link to a website where information should be uploaded or any other information which may be used to record delivery of a medicament.

The term window may be understood to encompass any transparent area of a housing. The transparent area may be achieved, for example, by application of an opaque label having a transparent area, use of different materials in manufacturing the housing or any other suitable means.

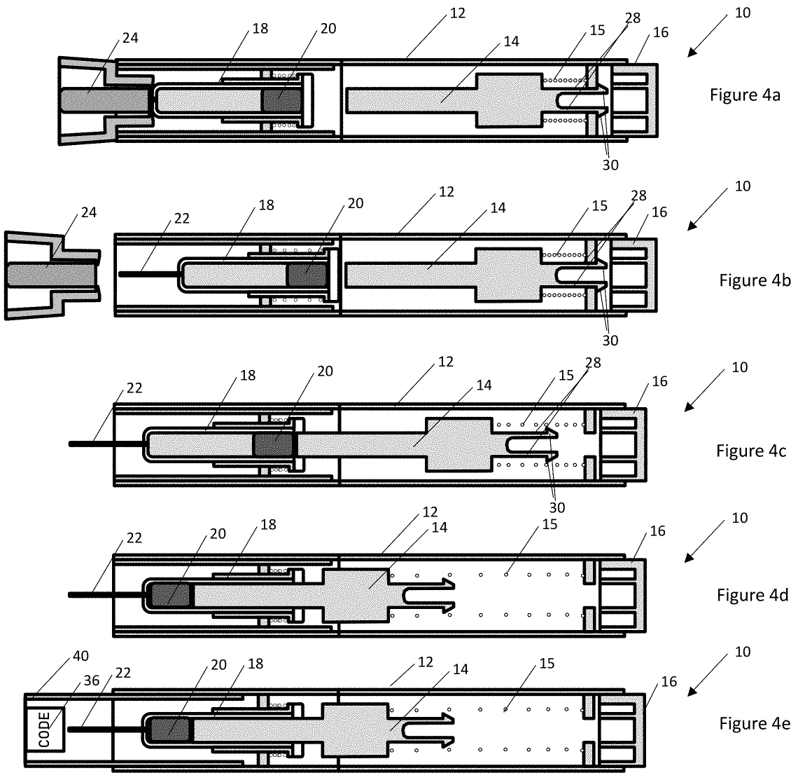

An injection device according to an embodiment of the present invention is described herein with reference to FIGS. 4a to 4e.

Like reference numerals have been used to denote features which are the same as FIG. 1. In FIGS. 4a to 4e the housing 12 is not provided with a window 38. Rather the information code 36 is provided on a needle shroud 40. The needle shroud 40 is contained within the housing 12 prior to and during injection.

The auto-injector may be activated as described with reference to FIGS. 1a to 1e. Once the injection is completed the needle shroud 40 extends from the housing thereby exposing the information code 36. This may be read as described previously with reference to FIGS. 1a to e.

The needle shroud 40 may be primed to move out of the housing in any suitable manner. For example, it may be configured to move out of the housing through pressure on the shroud 40 from the plunger 14. Alternatively it may be configured with a track and a compression spring exerting a force to move it out of the housing. Pressure on the shroud 40 from placing the auto-injector against a part of a body may cause the shroud 40 to move such that removal of the body allows the compression spring to force the shroud out of the body.

The skilled person will understand that although the auto-injector has been described as having a button which activates the device by releasing flexible arms from an interaction with projection from the housing any other suitable means may be used to activate the device. For example, a retractable shroud may cause the spring to be released or a sliding button on the side of the auto-injector.

Additionally, the device may be provided with drive means other than a compression spring. For example, the drive means may be a torsion spring, motor or any other suitable means configured to cause the plunger of the device to move to expel drug from the medicament container.

It will be understood by the skilled person that although the above referenced invention has been described with reference to an auto-injector it may be applied to any other injection device where operation of the device can cause a part of the device or syringe to move from being unseen to seen during operation of the device.

As will be understood by the skilled person, the medicament may be any substance suitable for injection. These substances include, but are not limited to drugs, vaccines, hormone, peptide or any other injectable substance.

Certain terminology is used in the following description for convenience only and is not limiting. The word “distal” refers to the front, or patient, end of the device. The word “proximal” refers to the rear, or syringe user, end of the device. The term “longitudinal”, with or without axis, refers to a direction on an axis through the device in the direction of the longest extension of the device. The term “radial” or “transverse/transversal”, with or without axis, refers to a direction generally perpendicular to the longitudinal direction, e.g. “radially outward” would refer to a direction pointing away from the longitudinal axis.

It should be understood that the detailed description and specific examples are given by way of illustration only, since various changes and modifications within the spirit and scope of the disclosure will become apparent to those skilled in the art from the description.

1. An injection device for receiving a medicament container comprising movable means, the injection device being configured such that before injection an information code is not visible and such that during or after injection the movable means moves to allow the code to be visible.

2. An injection device as claimed in claim 1 wherein the injection device comprises a housing including a transparent area, wherein the movable means moves from a position where the information code cannot be viewed through the transparent area to a position where the information code can be viewed through the transparent area.

3. An injection device as claimed in claim 2 wherein the movable means is a plunger and the information code is situated on the plunger, the plunger moving from a start position in which the information code cannot be viewed to a final position where the information code can be viewed.

4. An injection device as claimed in any one of claim 2 or 3 wherein the transparent area comprises a transparent area in a label applied to a housing or a transparent area in the material of the housing.

5. An injection device as claimed in any preceding claim wherein the movable means is a needle shroud and the information code is on the medicament container, the needle shroud moving from a position where it covers the information code in order that the information code cannot be viewed to a position where the information code can be viewed.

6. An injection device as claimed in claim 1 wherein the movable means comprises a needle shroud, the information code being carried on the needle shroud, wherein the needle shroud moves from a position where it is at least partially contained within a housing of the injection device and the information code is not visible to a position where the needle shroud extends from the housing and the information code is visible.

7. An injection device as claimed in any preceding claim wherein the injection device comprises an auto-injector

8. An injection device as claimed in any preceding claim wherein the information code comprises a bar code or a QR code.
